(19) United States
(12) Patent Application Publication
(10) Pub. No.: US 2024/0409569 A1
(43) Pub. Date: Dec. 12, 2024

(54) SOLID DOSAGE MINOR REDUCTION IN CRYSTALLIZATION OF STEVIA EXTRACT WITH LOW STEVIOL GLYCOSIDE CONTENT

(71) Applicant: CORN PRODUCTS DEVELOPMENT, INC., WESTCHESTER, IL (US)

(72) Inventors: Nan QI, Bridgewater, NJ (US); Christopher GREGSON, Bridgewater, NJ (US); Sarfaraz PATEL, Bridgewater, NJ (US)

(21) Appl. No.: 18/695,086

(22) PCT Filed: Oct. 6, 2022
(86) PCT No.: PCT/US2022/045865

§ 371 Date: Mar. 25, 2024

Related U.S. Application Data
(60) Provisional application No. 63/254,324, filed on Oct. 11, 2021.
(60) Provisional application No. 63/290,048, filed on Dec. 15, 2021.

(51) Int. Cl. C07H 1/08; A23L 27/30; C07H 15/256

(52) U.S. Cl. CPC C07H 1/08; A23L 27/36; C07H 15/256

(57) ABSTRACT

Through solid dosage minor reduction, the weight percentage of solids in an initial solution of crystallization is reduced in a controlled manner for crystallizing a stevia extract or remaining stevia extract starting material with low steviol glycoside content to obtain crystal steviol glycosides. The solid dosage minor reduction in the crystallization process imparts increased efficiencies to manufacturing processes and enables one or more benefits such as increased particle size distribution of the final crystal product, completion of one or more of solid product isolation processes (e.g., filtration and/or centrifugation) after crystallization, increased dewatering rate in solid product isolation processes, increased speed in subsequent solid drying, and enhanced product purities.

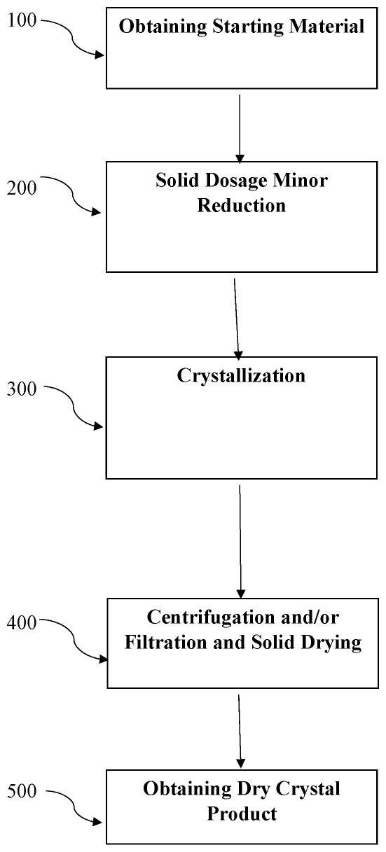

The present invention relates to crystallization of stevia extracts.

In many industries, crystallization is an important separation and purification process. It allows one to generate crystalline products, which also may be referred to as crystallization products or crystal products, that can be incorporated into end products such as those that are manufactured in the food and beverage industry, the pharmaceutical industry, and the cosmetics industry, as well as in many other industries.

When developing a process for crystallization, one needs to be mindful of the effect that the composition of the starting material can have on the crystallization process itself, subsequent processes, and final crystal products. For example, when trying to obtain a purified single steviol glycoside or a purified plurality of steviol glycosides from a starting material that is or contains a stevia extract that has a low steviol glycoside content, the particle sizes of the crystallization products are often small, which can cause subsequent filtration and/or centrifugation to take an undesirably long time or even to be unable to be completed. Additionally, even if one is able to complete filtration and/or centrifugation after a very long time, the purity of the crystalline product is often undesirably low.

Therefore, there is a need to develop new crystallization strategies for starting materials, e.g., feed materials, that have low steviol glycoside contents. The present invention is directed to this need.

The present invention provides new and non-obvious strategies for generating crystallization products from starting materials that have low steviol glycoside contents in order to obtain products that contain a purified single steviol glycoside or a purified plurality of steviol glycosides. The crystallization products of the present invention may flexibly be used in end products in which other crystalline steviol glycosides can be used. By way of non-limiting examples, these crystallization products, which comprise one or more steviol glycosides, may be used in food and beverage products, cosmetics, and pharmaceuticals.

In some embodiments, the present invention is directed to a method for making a crystallization product. The method comprises: (a) obtaining a starting material, wherein the starting material comprises, consists essentially of, or consists of solids and a solvent, wherein the solids comprise one or a plurality of steviol glycosides, the starting material is at a solid dosage of X wt. %, wherein solid dosage percentage is calculated as follows:

$$X = \frac{weight\_solid}{weight\_solid + weight\_solvent} \times 100$$

wherein, weight_solid is the combined weight of all solids in the starting material, and weight_solvent is the total weight of the solvent in the starting material, and the starting material has any one, two or all three of (i) a total amount of steviol glycosides that is less than or equal to 85% or less than or equal to 80% or less than or equal to 73% on a dry weight basis, (ii) Reb A in an amount of less than or equal to 56% on a dry weight basis, and (iii) Reb M in an amount of minimum 20% on a dry weight basis; (b) reducing the solid dosage of the starting material to form a material with a reduced solid dosage, wherein the reduced solid dosage is Y wt. % wherein Y is between 70% and 98% of X; (c) after step (b) subjecting the material with a reduced solid dosage to a crystallization process; and (d) obtaining a crystallization product comprising one or more crystals.

In some embodiments, the present invention is directed to a crystallization product that is produced by a method of the present invention.

In some embodiments, the present invention is directed to a use of a crystallization product of the present invention in a food, beverage, cosmetics, or pharmaceutical product.

Through various embodiments of the present invention, one may obtain one or more of the following benefits: increased product crystal size or crystal particle size distribution; reduced process time or increased process productivity in subsequent solid-liquid phase separation such as filtration and/or centrifugation; increased quality or purity of a final solid product, wherein the quality or purity is defined as the composition of one or more steviol glycosides in the solid product on a dry weight basis; and reduced process time or increased process productivity in subsequent solid product drying. The phrase “dry weight basis” refers to the weight of a dry material after all liquid has been removed.

Various embodiments of the present invention are particularly advantageous when high-quality starting materials, such as starting materials with high steviol glycoside content, are not available or are not feasible to use because of their high prices. Further, in some embodiments, the present invention allows for the use of low-quality starting materials with low steviol glycoside content to make valuable products. In some embodiments, these products from low-quality starting materials can be made at a higher production rate with a product quality that is better than or at least equivalent to products made from high-quality starting materials.

### BRIEF DESCRIPTION OF THE FIGURE

The FIGURE is a flowchart that is a representation of a method of the present invention.

Reference will now be made in detail to various embodiments of the present invention, an example of which is illustrated in the accompanying FIGURE. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context. Further, headers are provided for the convenience of the reader and do not limit the scope of any of the embodiments described herein.

### Starting Materials

A “starting material” of the present invention is a material from which a crystallization product that contains one or a plurality of steviol glycosides can be obtained. Thus, the starting material itself will comprise one or a plurality of steviol glycosides. A starting material of the present invention may also be referred to as a feed material or a raw material. The starting material may comprise, consist essentially of, or consist of both solids (which may include both steviol glycosides and other solid materials) and one or more solvents.

The term “steviol glycoside” refers to all compounds with a general structure of the steviol backbone conjugated to any number, combination, or orientation of saccharides (glucose, rhamnose, xylose, fructose, arabinose, galactose and deoxyglucose), such as those occurring in the leaves of Stevia rebaudiana Bertoni or its genetically modified varieties. The term “steviol glycoside” refers to both steviol glycosides that are now known and steviol glycosides that come to be known and recognized by persons of ordinary skill in the art as falling within this class of compounds.

Examples of steviol glycosides include but are not limited to rebaudioside A (Reb A), rebaudioside B (Reb B), rebaudioside C (Reb C), rebaudioside D (Reb D), rebaudioside E (Reb E), rebaudioside F (Reb F), rebaudioside M (Reb M), rubusoside, dulcoside A, rebaudioside I, rebaudioside Q, rebaudioside N, rebaudioside O, steviosides such as 1,2-stevioside and 1,3-stevioside, steviolbiosides such as steviol-1,2-bioside and steviol-1,3-bioside, steviol-13-O-glucoside (13-SMG), and steviol-19-O-glucoside (19-SMG). The starting materials may contain one or more of the aforementioned steviol glycosides.

In some embodiments, the starting material comprises, consists essentially of, consists of, or is derived from a stevia extract. Thus, the starting material may, for example, be obtained directly from a Stevia rebaudiana Bertoni plant, or after some processing of either an extract of the plant or the plant itself. Technologies for obtaining stevia extracts are well-known to persons of ordinary skill in the art. For example, one may extract steviol glycosides from stevia leaves using water or water-organic solvent mixtures. Optionally, the leaves may be pretreated by, for example, fatty acid extraction, clarification, de-coloration, and ash removal. In some embodiments, the starting material for the present invention is or comprises a by-product material from a broader process. For example, the starting material may contain a set of steviol glycosides that comprises, consists essentially of, or consists of a plurality of steviol glycosides that have been obtained as a by-product from an earlier process. The by-product may, for example, be obtained from any separation and purification processes, and optionally have been spray dried before being used as a starting material.

In some embodiments, the starting material comprises at least one of Reb A, Reb B, Reb C, Reb D, Reb F, dulcoside A, rubusoside, stevioside, steviolbioside, and Reb M.

In some embodiments, the starting material comprises all of Reb A, Reb B, Reb C, Reb D, Reb F, dulcoside A, rubusoside, stevioside, and steviolbioside.

In some embodiments, within the starting material the steviol glycoside content due to steviol glycosides other than Reb A, Reb B, Reb C, Reb D, Reb F, dulcoside A, rubusoside, stevioside, and steviolbioside is less than 5.0%, less than 4.0%, less than 3.0%, less than 2.0%, less than 1.0%, less than 0.5%, less than 0.1% or is 0% on a dry weight basis.

In some embodiments, the starting material comprises Reb A.

In some embodiments, the starting material comprises Reb B.

In some embodiments, the starting material comprises Reb C.

In some embodiments, the starting material comprises Reb D.

In some embodiments, the starting material comprises Reb F.

In some embodiments, the starting material comprises Reb M.

In some embodiments, the starting material comprises dulcoside A.

In some embodiments, the starting material comprises rubusoside.

In some embodiments, the starting material comprises stevioside.

In some embodiments, the starting material comprises steviolbioside.

### Steviol Glycoside Content

A starting material may be defined by its “steviol glycoside content.” This characteristic of the starting material may provide information as to the composition of any one steviol glycoside, any plurality of steviol glycosides individually or cumulatively, or all steviol glycosides individually or cumulatively in the starting material.

By way of non-limiting examples, in some embodiments a starting material's steviol glycoside content may refer to: the composition of any individual steviol glycoside from the group consisting of Reb A, Reb B, Reb C, Reb D, Reb F, Reb M, dulcoside A, rubusoside, stevioside, or steviolbioside; the composition of a plurality of steviol glycosides from the group consisting of Reb A, Reb B, Reb C, Reb D, Reb F, Reb M, dulcoside A, rubusoside, stevioside, and steviolbioside; the cumulative composition of a plurality of steviol glycosides; or the cumulative composition of all the steviol glycosides within the starting material. When referring to the cumulative composition of all of the steviol glycosides in a starting material, one may refer it to “total steviol glycoside” (“TSG”) content.

For various embodiments of the present invention, the starting material has what may be referred to as a “low steviol glycoside content.” A low steviol glycoside content denotes that the composition (on a dry weight basis) of one steviol glycoside, or a plurality of steviol glycosides, or all steviol glycoside cumulatively, is less than the composition of the same steviol glycosides in a raw material that is normally or previously used in industrial crystallization to generate desirable crystal products. These latter starting materials have higher compositions or concentrations of steviol glycosides and may be referred to as having a “high steviol glycoside content.”

A starting material with a high steviol glycoside content may be acceptable for use in commercial production to make desirable crystal products, and has a certain or certain range of solid dosage level in a crystallization process. Further, its commercial production has an acceptable process time, production rate, product yields, and product purities.

In some embodiments in which the starting material has a low steviol glycoside content, its TSG content (i.e., the cumulative composition of all steviol glycosides) is less than or equal to 85 wt. %, less than or equal to 80 wt. %, or less than or equal to 73 wt. % on a dry weight basis.

In some embodiments in which the starting material has a low steviol glycoside content, its steviol glycoside content of the specific steviol glycoside Reb A is less than or equal to 56 wt. % on a dry weight basis.

### Solid Dosage

“Solid dosage” refers to the relative amount of solid and solvent used in a crystallization process and is calculated according to the following formula:

$$solid dosage = \frac{weight\_solid}{weight\_solid + weight\_solvent} \times 100 \% \qquad Formula I$$

wherein “weight_solid” is the weight of the dry solids and “weight_solvent” is the weight of the solvent. By way of non-limiting examples, in some embodiments, the starting materials used in the present invention may have a solid dosage of 12%-40%, 14%-36%, 14-20%, 16-20%, 17-19%, or 32%-36%.

The parameter of solid dosage is particularly relevant when a starting material is subjected to a crystallization process. As the formula above notes, one may define a mixture or solution as having a solid dosage of a defined percentage, and it may be referred to as “in a crystallization process” because the solid is to be fully dissolved by a solvent in the beginning of a crystallization process. “Weight_solid” and “weight_solvent” refer to the cumulative weights of all solid components, not only steviol glycosides, and all solvent components, respectively. Thus, a starting material may be described by its “solid dosage” or its “solid dosage of X % in a crystallization process.”

### Solid Dosage Minor Reduction

When starting materials are changed with respect to their steviol glycoside contents from high to low but are subjected to the same crystallization processes at the same solid dosage level, often these starting materials either cannot be used to make desirable products or cause one or more production processes such as filtration, centrifugation, or solid drying to take unacceptably long times to complete. To be able to make desirable products from these low steviol glycoside content starting materials, one may subject these starting materials to a solid dosage minor reduction in their crystallization processes.

A “solid dosage minor reduction” refers to a relatively small amount of reduction of solid dosage. It means that in the beginning of or prior to crystallization, one dissolves or dilutes the starting material such that there is a smaller quantity of total solids, including all steviol glycosides and other solids, per volume of solvent.

In some embodiments, the reduction is by no more than 50% of the solid dosage initial value, i.e., to a value that is at least 50% of the starting solid dosage. In some embodiments of the present invention, there is a solid dosage minor reduction 200 of between 0.1 and 50.0% of the initial solid dosage value. Thus, if the initial solid dosage for a starting material 100 with a low steviol glycoside content has a value of X wt. % and the new solid dosage after the minor reduction has a value of Y wt. %, Y is between 50.0% and 99.9% of X, or between 70.0% and 98.0% of X, or between 75.0% and 95.0% of X, or between 80.0% and 90.0% of X.

As persons of ordinary skill in the art will recognize, the aforementioned resulting Y wt. % corresponds to reductions of between 0.1% and 50%; 2% and 30%; 5% and 25%; and 10% and 20%. The aforementioned relationships between X and Y are defined relative to each other, i.e., Y as a percent of X. One can also define a solid dosage minor reduction by reference to an absolute number. Thus, by way of a non-limiting example, solid dosage minor reduction may be from X wt. % to Y wt. %, wherein Y is between X—0.5 and X—15.0, or Y is between X—1.0 and X—5.0.

A solid dosage reduction may be accomplished by rendering a solution more dilute, e.g., adding more of the solvent. Examples of solvents include but are not limited to water, methanol, ethanol, propanol, acetone, acetic acid, acetonitrile, tetrahydrofuran, pyridine, methyl acetate, ethyl acetate, methylene chloride, ethyl ether, chloroform, dioxane, carbon tetra-chloride, toluene, benzene, and cyclohexane, as well as combinations thereof.

Solid dosage minor reduction may flexibly be incorporated into many crystallization processes.

Some of the methods of present invention begin by obtaining a starting material 100, which may be a crude stevia extract or a remaining stevia extract. Each starting material has at two parameters of importance: (1) steviol glycoside content; and (2) solid dosage used in a crystallization process.

The methods of the present invention may be used to obtain crystallization products that comprise, consist essentially of, or consists of any individual steviol glycoside, e.g., any one of the steviol glycosides referenced in this disclosure. Alternatively, the crystallization products may comprise, consist essentially of, or consist of a plurality of steviol glycosides. The steviol glycoside(s) in the crystallization products are also present in the starting material. These crystallization products may be obtained under conditions that lead to desirable levels of purity, e.g., at least 80%, at least 85%, at least 90%, or at least 95%.

In some embodiments, a starting material is used in order to obtain a crystallization product with greater than or equal to 95% TSG on a dry weight basis. This starting material may have a TSG content of less than or equal to 85%, less than or equal to 80%, less than or equal to 73%, less than or equal to 70%, less than or equal to 65%, less than or equal to 60%, less than or equal to 55%, less than or equal to 50%, less than or equal to 45%, less than or equal to 40%, less than or equal to 35%, less than or equal to 30%, or less than or equal to 25% on a dry weight basis. In some embodiments, the TSG is between 20% and 73%, or between 20% and 50%, or between 30% and 40%, or between 58% and 73% of the starting material on a dry weight basis. These starting materials may be subjected to a solid dosage minor reduction during the crystallization process according to the present invention.

In some embodiments, another starting material is used in order to obtain a crystallization product with greater than or equal to 95% Reb A on a dry weight basis. In these starting materials, the Reb A content is less than or equal to 56%, less than or equal to 52%, less than or equal to 45%, less than or equal to 35%, less than or equal to 25%, less than or equal to 15%, or less than or equal to 5% on a dry weight basis. In some embodiments, the Reb A content in the starting materials may be present in an amount of 20% to 56%, or 30% to 50% of the starting material on a dry weight basis. These starting materials may be subjected to a solid dosage minor reduction during a crystallization process.

In some embodiments, the starting material comprises Reb A and additionally or alternatively, comprises at least one of, a plurality of, or all of Reb B, Reb C, Reb D, Reb F, dulcoside A, rubusoside, stevioside, and steviolbioside. In some embodiments, the TSG content that is attributable to the set of the aforementioned steviol glycosides (including Reb A) or a subset of those substances if not all are present in the starting material is at least 70%, at least 75%, at least 80%, at least 90%, or at least 95% of the total dry weight of the starting material. By way of non-limiting examples, when present, any or each of these materials may be present in the following amounts on dry weight basis: Reb A: 20% to 67%; Reb B: 0.5% to 5.0%; Reb C: 4.0% to 8.5%; Reb D: 1.5% to 3.0%; Reb F: 0.5% to 2.0%; dulcoside A: 0.1% to 4.0%; rubusoside: 0.5% to 14.0%; stevioside: 5.0% to 16.0%; and steviolbioside: 0.1% to 1.0%. These starting materials may be subjected to a solid dosage minor reduction in crystallization processes.

By applying the solid dosage minor reduction method of the present invention during a crystallization process to a starting material that has a low steviol glycoside content, one may efficiently be able to obtain the desired crystallization products of steviol glycoside(s) from that starting material. In various embodiments, this method imparts one or more of the following benefits: shortened length of process time in subsequent filtration and/or centrifugation and solid drying, increased purity of the crystallization product, and increased particle size distribution of the crystallization product. One or more of these process and product attributes may be comparable to or even better than those observed when applying crystallization processes to starting materials that have a high steviol glycoside content.

### Temperature

The various embodiments of the present invention may flexibly be employed at any temperature profiles that are currently being used in crystallization processes. Therefore, in some embodiments, crystallization with solid dosage minor reduction and subsequent filtration or/and centrifugation processes may be carried out at ambient temperature. In other embodiments, heat is introduced, thereby raising the temperature of the system or heat is removed, thereby lowering the temperature of the system.

Additionally, the temperature may be kept constant throughout the process of the present invention or it may vary. Further, when the temperature varies, the variation may, during one or more time periods, be regular, irregular, increasing, decreasing, or both increasing and decreasing. For example, one or more of the steps of the processes described herein may be conducted at elevated temperatures. In one non-limiting example, the solid dosage minor reduction described herein is obtained at ambient temperate or at a temperature less than or equal to 55° C., and during one or both of subsequent crystallization and filtration, the temperature is elevated to a temperature greater than 55° C. or greater than 60° C. or greater than 65° C. or greater than 70° C., and in some embodiments, up to 90° C. or up to 85° C. or up to 80° C.

### Industrial Processes

The methods of the present invention may be used one or more times in the crystallization process of obtaining desirable crystal products. For example, when making a sweetener product comprising purified steviol glycosides, one may follow the generally known steps as shown below:
1. First crystallization step:
   Crude stevia extract→Reb A+First by-product liquid stream
2. Spray drying step:
   First by-product liquid stream→Remaining stevia extract
3. Second crystallization step:
   Remaining stevia extract→Purified steviol glycosides+Second by-product liquid stream
4. Blending step:
   Purified steviol glycosides+Reb A→Stevia sweetener product

During prior art processes that correspond to Step (3) as described above, the resulting purified steviol glycosides from the starting materials with low steviol glycoside content were often not within desired product specifications. One could make use of those products by blending them with Reb A as shown in Step (4) in order to reach the desired Reb A composition in the final product. However, that blending step can be cumbersome and costly. With methods of the present invention, the second crystallization products (“purified steviol glycosides”) are of sufficient quality that Step (4), the final step of blending with Reb A, can be avoided or the amount of Reb A required in blending can be significantly reduced.

When implementing the present invention, in e.g., either Step (1) or Step (3), the total rejection or minimization of impurities in a product of the crystallization process 300 is desirable. What is an impurity may be defined relative to which compounds are desired to be in the crystallization product. Thus, by way of a non-limiting example, in Step (1) noted above, because the crystallization product is Reb A, the impurities in the “first by-product liquid stream” will include all other non-Reb A steviol glycosides, all non-steviol glycoside compounds, polymers, color bodies, proteins, and other compounds and materials to the extent that they are present. In some embodiments, the crude stevia extract in Step (1) contains 35-51 wt. % impurities on dry weight basis. Removal of impurities can be accomplished through e.g., selection of the appropriate solvent, temperature profile, and agitation conditions, which are known to persons of ordinary skill in the art for developing crystallization processes to obtain products that contain desired steviol glycosides.

By way of another non-limiting example, in Step (3) noted above, the crystal product is a mixture of crystalline Reb A and other crystalline steviol glycosides. Thus, the impurities may, for example, include non-steviol glycosides, color bodies, proteins, and other compounds and materials to the extent that they are present. In some embodiments, when the crystallization starting materials are the “remaining stevia extract” in Step (3), they contain 19-51% impurities on dry weight basis.

Although one wishes to remove all impurities, in some instances, residual impurities remain in the crystallization product. In some embodiments, the impurities within a crystallization product are less than 5 wt. %, less than 4 wt. %, less than 3 wt. %, less than 2 wt. %, less than 1 wt. %, or less than 0.5 wt. % of the crystallization product on a dry weight basis.

### Recovering and Obtaining Crystallization Products

Following the crystallization to which the solid dosage minor reduction is applied, the crystallization end slurry may undergo any subsequent processes known to persons of ordinary skill in the art to obtain final crystal products 500. By way of non-limiting examples, these processes may be one, some, or all of filtration, centrifugation, solids drying 400, and solid dry milling.

### Uses

The crystallization products of the present invention may, for example, be used in food and beverage, cosmetic, or pharmaceutical applications. In some embodiments, one may combine a crystallization product obtained by any of the various embodiments of the present invention with other ingredients to make a food or beverage product. In some embodiments, these crystallization products are first combined with other steviol glycosides (e.g., Reb A), prior to, or while being, combined with other ingredients of the end products. In other embodiments, the crystallization products are the sole source of steviol glycosides in the end products. By way of non-liming examples, one may combine ingredients by mixing, blending, or any other methods that are now known, or that come to be known, for combining crystallization products of the present invention with other ingredients of food and beverage products. In some embodiments, the crystallization products of the present invention are used as sweeteners in food and beverage products.

Examples of food products in which the crystallization products of the present invention may be used include, but are not limited to, confections, condiments, chewing gum, frozen foods, canned foods, soy-based products, salad dressings, mayonnaise, vinegar, ice cream, cereal compositions, baked goods, dairy products such as yogurts, and tabletop sweetener compositions.

Examples of beverages in which the crystallization products of the present invention may be used include, but are not limited to, ready-to-drink products that are carbonated (e.g., colas or other soft drinks, sparkling beverages, and malts) or non-carbonated (e.g., fruit juices, nectars, vegetable juices, sports drinks, energy drinks, enhanced water, coconut waters teas, coffees, cocoa drinks, beverages containing milk, beverages containing cereal extracts, smoothies, and alcoholic beverages), as well as powdered beverage products that are to be combined with a liquid base such as water, milk, or club soda.

The crystallization products of the present invention may also be used in cosmetic compositions, dental compositions, and pharmaceutical compositions.

Subject matter contemplated by the present disclosure is set out in the following numbered embodiments:
1. A method for making a crystallization product comprising:
   (a) obtaining a starting material, wherein the starting material comprises solids and a solvent, wherein the solids comprise one or a plurality of steviol glycosides, and the starting material is at a solid dosage of X wt. %, wherein

$$X = \frac{weight\_solid}{weight\_solid + weight\_solvent} \times 100 ,$$

weight_solid=combined weight of all solids in the starting material, and weight_solvent=total weight of the solvent in the starting material; and the starting material has any one, two or all three of (i) a total composition of steviol glycosides of less than or equal to 85% on a dry weight basis, (ii) Reb A in an amount of less than or equal to 56% on a dry weight basis, and (iii) Reb M in an amount of minimum 20% on a dry weight basis; (b) reducing the solid dosage of the starting material to form a material with a reduced solid dosage, wherein the reduced solid dosage is Y wt. % wherein Y is between 70% and 98% of X; (c) after step (b) subjecting the material with a reduced solid dosage to a crystallization process; and (d) obtaining a crystallization product comprising one or more crystals.
2. The method according to embodiment 1, wherein Y is between 75% and 95% of X.
3. The method according to embodiment 2, wherein X is between 14 and 36.
4. The method according to embodiment 3, wherein X is between 32 and 36.
5. The method according to embodiment 3, wherein X is between 14 and 20.
6. The method according to embodiment 5, wherein X is between 17 and 19.
7. The method according to any of embodiments 1 to 6, wherein the starting material comprises a plurality of steviol glycosides.
8. The method according to embodiment 7, wherein in the starting material, the plurality of steviol glycosides comprises at least one of Reb A, Reb B, Reb C, Reb D, Reb F, dulcoside A, rubusoside, stevioside, steviolbioside, and Reb M.
9. The method according to embodiment 8, wherein in the starting material, the plurality of steviol glycosides comprises each of Reb A, Reb B, Reb C, Reb D, Reb F, dulcoside A, rubusoside, stevioside, and steviolbioside.
10. The method according to any of embodiments 1 to 9, wherein in the starting material, the steviol glycoside content due to steviol glycosides other than Reb A, Reb B, Reb C, Reb D, Reb F, dulcoside A, rubusoside, stevioside, and steviolbioside is less than 5.0% or is 0% on a dry weight basis.
11. The method according to embodiment 10, wherein in the starting material, the steviol glycoside content due to steviol glycosides other than Reb A, Reb B, Reb C, Reb D, Reb F, dulcoside A, rubusoside, stevioside, and steviolbioside is less than 2.0% or is 0% on a dry weight basis.
12. The method according to any of embodiments 1 to 11, wherein the starting material comprises Reb M.
13. The method according to embodiment 1, wherein the starting material comprises a crude stevia extract.
14. The method according to embodiment 1, wherein the crystallization process is a second crystallization process and the crystallization product is a second crystallization product, and the method further comprises subjecting a stevia extract to a first crystallization process thereby obtaining a first crystallization product and a by-product liquid stream as a mother liquor; spray drying the by-product liquid stream, thereby obtaining a set of remaining steviol glycosides; and using the set of remaining steviol glycosides as said starting material.
15. The method according to embodiment 14, wherein the first crystallization product comprises Reb A or Reb D or a combination of thereof.
16. The method of any of embodiments 1 to 15, wherein the starting material has both (i) the total steviol glycoside amounts of less than or equal to 73% on a dry weight basis, and (ii) the Reb A is an amount of less than or equal to 56% on a dry weight basis.
17. The method according of any of claims 1 to 15, wherein the total composition of steviol glycosides is less than 80%.
18. The method according to claim 17, wherein the total composition of steviol glycosides is less than 73%.
19. The method according any of claims 1 to 18, wherein in the starting material Reb M is present in an amount of minimum 20% on a dry weight basis.
20. The method of any of claims 1 to 19, wherein step (c) is conducted at a temperature of greater than 55° C.
21. The method of any of claims 1 to 20, wherein step (d) comprises filtering and said filtering occurs at a temperature of greater than 55° C.
22. A method of making a food or beverage product comprising obtaining a crystallization product according to any of embodiments 1 to 21 and combining said crystallization product with one or more food, beverage or sweetener ingredients.
23. A crystallization product obtained in accordance with any one of embodiments 1 to 21.
24. A use of the crystallization product obtained in accordance with any of embodiments 1 to 21 to produce a food or beverage product.

### EXAMPLES

In examples 1, 2, and 3, the starting materials were the “remaining stevia extracts” after significant amounts of Reb A were separated out from crude stevia extracts through the first crystallization as described in paragraph [0059]. The steviol glycoside content of these “remaining stevia extracts” are shown in Table I.

TABLE I
Steviol Glycoside Content of Starting Materials (on dry weight basis) Used in Examples 1, 2, and 3

| | Reb | Stevioside, | Dulcoside | Reb | Reb | Reb | Reb | SteviolBioside,, | Rubusoside, | TSG, |
|---|---|---|---|---|---|---|---|---|---|---|
| Sample | A, % | % | A, % | B, % | C, % | D, % | F, % | % | % | % |
| A, N | 32.7 | 19.7 | 0.2 | 1.7 | 15.0 | 8.3 | 2.0 | 0.3 | 0.7 | 80.6 |
| B | 32.4 | 13.8 | 0.4 | 2.5 | 11.8 | 2.2 | 2.4 | 0.4 | 7.5 | 73.4 |
| C, D, E, | 24.4 | 10.8 | 1.4 | 4.6 | 10.1 | 3.4 | 1.4 | 1.1 | 5.3 | 62.5 |
| H, K, O, R | | | | | | | | | | |
| F, G, I, | 26.8 | 5.2 | 3.8 | 2.9 | 7.0 | 1.1 | 1.0 | 0.5 | 12.0 | 60.2 |
| L, P, Q | | | | | | | | | | |
| J, M | 14.8 | 12.6 | 0.6 | 6.3 | 5.6 | 2.0 | 2.2 | 3.1 | 2.5 | 49.7 |

### Example 1

“The second crystallization” described in paragraph [0059] was performed on each starting material (Samples A, B, C, D, E, F, G) to obtain purified steviol glycosides with the product quality specification of greater than or equal to 95% TSG. In this example, 35.0 g of a starting material as dry solid and 65.0 g of methanol were added into a crystallizer. Thus, the starting solid dosage for crystallization was 35%. The mixture was dissolved during agitation. Crystallization included the increase of temperature and addition of a seed.

After the crystallization, dead end filtration was performed. (The same filtration set was used for the experiments done in Example 1, in Example 2, and on Samples (A, C, I, J, K, L, M) in a 0.2 L crystallizer in Example 3. A similar but larger filtration set was used for Samples (N, O, P, Q, R) in a 2.0 L crystallizer in Example 3.) The weight of filtrate that was collected and the time that was taken to filter for each filtration were recorded. The filtration rate was calculated as the weight of filtrate that was collected divided by the filtration time.

Table II provides a summary of the effects on the filtration rate due to the different steviol glycoside contents of starting materials. For each crystallization experiment described here, the solid dosage was: (1) kept at a baseline level of 35%, which was the same as the crystallization of high steviol glycoside starting materials (greater than 73% TSG); or (2) subjected to an increase to greater than 35%.

TABLE II
Low TSG Content Starting Materials Subjected to
Solid Dosage Increases in the 2nd Crystallization

| | Starting | Starting | Solid | |
|---|---|---|---|---|
| | Material | Material | Dosage, | Filtration |
| Sample | Reb A, % | TSG, % | % | Rate, g/s |
| A | 32.7 | 80.6 | 34.4-35.0 | 0.6-0.7 |
| B | 32.4 | 73.4 | 35.0 | 0.7-0.8 |
| C | 24.4 | 62.5 | 35.0 | 0.4-0.6 |
| D | 24.4 | 62.5 | 38.0 | 0.1 |
| E | 24.4 | 62.5 | 39.4 | 0.1-0.2 |
| F | 26.8 | 60.2 | 35.1 | 0.6 |
| G | 26.8 | 60.2 | 37.2 | 0.2 |

Sample A is a starting material with a high steviol glycoside content (80.6% TSG). Its crystallization was conducted as a baseline experiment with solid dosage at 34.4-35.0%. Its filtration rate in the end slurry and product purity (TSG %, Reb A %) were acceptable and set as the benchmark to be compared with the results from other experiments. The TSG (73.4%) content of Sample B is also considered high. For each of Samples A and B, the solid dosage was ˜35%, and the filtration rates (0.6-0.8, g/s) were regarded as high.

In Sample C, the TSG (62.5%) is less than the TSG of Samples A and B, but the solid dosage was kept the same at 35.0% in crystallization. The relatively low TSG content of Sample C correlated with a low filtration rate (0.4-0.6, g/s) despite Sample C being subjected to a solid dosage similar to that of Samples A and B. Samples A, B, and C show that at the same solid dosage ˜35.0%, the low steviol glycoside (here referring to TSG) content in the starting material led to a low filtration rate after crystallization.

Samples D and E were from the same starting material as Sample C. For Sample D's crystallization, the solid dosage was increased to 38.0%. For Sample E's crystallization, the solid dosage was further increased to 39.4%. As Table II shows, the filtration rates (0.1-0.2, g/s) for Samples D and E were dramatically lower than the filtration rate (0.4-0.6, g/s) for Sample C. Thus, for the same low steviol glycoside (i.e., TSG) content starting material, high solid dosages led to low filtration rates.

Samples F and G were from the same starting material with TSG of 60.2% and 26.8% Reb A. For Sample F, the solid dosage was 35.1%. For Sample G, the solid dosage was increased from 35.1% to 37.2%. Both samples were subjected to the crystallization process. The filtration rate (0.2, g/s) for Sample G was greatly lower than the filtration rate (0.6, g/s) for Sample F. For the same starting material with a low steviol glycoside (TSG) content, again high solid dosage led to low filtration rate.

This example shows that the use of a starting material with a low steviol glycoside (here TSG) content leads to a decrease in filtration rate. Increasing the solid dosage for a starting material with a low steviol glycoside content was previously believed to be able to increase the TSG concentrations in the initial solution of crystallization to the same high levels as those from the starting materials with a high steviol glycoside content, and thus to be able to achieve the same high filtration rates as from the starting materials with a high steviol glycoside content. Surprisingly, increasing the solid dosages of those starting materials with a low steviol glycoside content doesn't increase, and instead decreases, the filtration rates.

### Example 2

Sample H and Sample C were from the same starting material, which had a low steviol glycoside (TSG) content of 62.5%. Both Samples H and C were subjected to the same crystallization process as described in Example 1, but at different solid dosage levels. Sample C had a baseline solid dosage level of 35.0%. Sample H was subjected to a solid dosage reduction from 35.0% to 26.0%. The filtration rate (1.0-1.1, g/s) of Sample H was faster than the filtration rate (0.4-0.6 g/s) of Sample C. Moreover, the filtration rate (1.0-1.1, g/s) from Sample H was faster than the filtration rate (0.6-0.7, g/s) from Sample A, which had much higher steviol glycoside content (80.6% TSG) than Sample H (62.5% TSG). Thus, for the same starting material with a low steviol glycoside content, solid dosage reduction led to a high filtration rate. Table III summarizes these results.

TABLE III
Low TSG Content Starting Materials Subjected to
Solid Dosage Decrease in the 2nd Crystallization

| | Starting | Starting | Solid | | Product |
|---|---|---|---|---|---|
| | Material | Material | Dosage, | Filtration | Yield, |
| Sample | Reb A, % | TSG, % | % | Rate, g/s | % (db) |
| A | 32.7 | 80.6 | 34.4-35.0 | 0.6-0.7 | 34-46 |
| C | 24.4 | 62.5 | 35.0 | 0.4-0.6 | >24-34 |
| H | 24.4 | 62.5 | 26.0 | 1.0-1.1 | 14-15 |

### Example 3

The same crystallization process of Examples 1 and 2 was conducted on different starting materials (Samples I, J, K, L, M, N, O, P, Q, R) at various low steviol glycoside (i.e., TSG) contents with solid dosages that were subjected to a minor reduction, from ˜35% to ˜31%. Experiments were performed on two scales, as in 0.2 L and 2.0 L crystallizers, respectively. The results are summarized in Tables IV and V. The experiments were repeated for a few times; therefore, some data are reported as in ranges.

TABLE IV
Low TSG Content Starting Materials Subjected to Solid Dosage Minor
Reduction in the 2^{nd} Crystallization: in 0.2 Liter Crystallizer

| | Starting | Solid | | Product | Product | Product | | Product |
|---|---|---|---|---|---|---|---|---|
| | Material | Dosage, | Filtration | Yield, % | TSG, % | Reb A, % | Product TSG | Reb A |
| Sample | TSG, % | % | Rate, g/s | (db) | (db) | (db) | Recovery % | Recovery % |
| A | 80.6 | 34.4-35.0 | 0.6-0.7 | 34-46 | 90-94 | 42-44 | 39-51 | 45-57 |
| C | 62.5 | 35.0 | 0.4-0.6 | >24-34 | <87-90 | 47-53 | 35-48 | 53-66 |
| I | 60.2 | 35.1 | 0.6 | >15 | <86 | 67 | 22 | 40 |
| J | 49.7 | 35.0 | 0.4-0.5 | >15 | <86 | 52 | 26 | 54 |
| K | 62.5 | 29.7-30.8 | 0.9-1.0 | 18-24 | 93 | 62 | 27 | 48 |
| L | 60.2 | 30.5 | 0.8 | 11-12 | 93 | 80 | 18 | 38 |
| M | 49.7 | 31.7 | 1.0-1.1 | 12 | 90 | 56 | 21 | 46 |

TABLE V
Low TSG Content Starting Materials Subjected to Solid Dosage Minor
Reduction in the 2^{nd} Crystallization: in 2.0 Liter Crystallizer

| | Starting | Solid | | Product | Product | Product | | Product |
|---|---|---|---|---|---|---|---|---|
| | Material | Dosage, | Filtration | Yield, % | TSG, % | Reb A, % | Product TSG | Reb A |
| Sample | TSG, % | % | Rate, g/s | (db) | (db) | (db) | Recovery % | Recovery % |
| N | 80.6 | 35.0 | 0.6 | 31 | 92 | 46 | 35 | 43 |
| O | 62.5 | 35.0 | 0.3 | 24 | 88 | 56 | 34 | 56 |
| P | 60.2 | 35.1 | 0.4 | 20 | 84 | 66 | 28 | 52 |
| Q | 60.2 | 30.5 | 1.8 | 13 | 92 | 77 | 20 | 40 |
| R | 62.5 | 30.9 | 1.1 | 20 | 92 | 62 | 29 | 51 |

Within Tables IV and V:
“Product TSG, % (db)” refers to product purity indicated as TSG % in final solid product on dry weight basis.
“Product Reb A, % (db)” refers to product purity indicated as Reb A % in final solid product on dry weight basis.

Comparing samples A, C, and K, and comparing N, O, and R, one sees that from the same starting material (Samples C, K, O, R) with low steviol glycoside content (62.5% TSG), solid dosage minor reduction from 35% (C, O) to ˜30-31% (K, R) leads to remarkably higher filtration rates (from 0.4-0.6 g/s for C increased to 0.9-1.0 g/s for K, from 0.3 g/s for O increased to 1.1 g/s for R) and higher product purities (from <87-90% TSG and 47-53% Reb A for C increased to 93% TSG and 62% Reb A for K, from 88% TSG and 56% Reb A for O increased to 92% TSG and 62% Reb A for R). Furthermore, the filtration rates and product purities of Reb A from the solid dosage minor reduction of present invention are even higher than those from the starting material with a high steviol glycoside content (80.6% TSG for Samples A and N) without solid dosage reduction, e.g., 0.9-1.0 g/s filtration rate and 62% Reb A for K, which are higher than 0.6-0.7 g/s filtration rate and 42-44% Reb A for A; 1.1 g/s filtration rate and 62% Reb A for R, which are higher than 0.6 g/s filtration rate and 46% Reb A for N. The product purities of TSG from the solid dosage minor reduction are at a similar level to those from starting materials with a high steviol glycoside content (80.6% TSG for Samples A and N) without solid dosage reduction, i.e., 93% TSG for K, which is similar to 90-94% TSG for A; 92% TSG for R, and 92% TSG for N.

Comparing A, I, and L, and comparing N, P, and Q, one sees that for the same starting material (Samples I, L, P, Q) with low steviol glycoside content (60.2% TSG), solid dosage minor reduction from ˜35% (I, P) to ˜31% (L, Q) remarkably improves filtration rates (from 0.6 g/s for I increased to 0.8 g/s for L, from 0.4 g/s for P increased to 1.8 g/s for Q) and improves product purities (from <86% TSG and 67% Reb A for I increased to 93% TSG and 80% Reb A for L, from 84% TSG and 66% Reb A for P increased to 92% TSG and 77% Reb A for Q). Furthermore, the filtration rates and product purities of Reb A with solid dosage minor reduction are higher than those from the starting material with a high steviol glycoside content (80.6% TSG, for Samples A and N) without solid dosage reduction, e.g., 0.8 g/s filtration rate and 80% Reb A for L, which are higher than 0.6-0.7 g/s filtration rate and 42-44% Reb A for A; 1.8 g/s filtration rate and 77% Reb A for Q, which are higher than 0.6 g/s filtration rate and 46% Reb A for N. The product purities of TSG from solid dosage minor reduction are at a level similar to the level from the starting material with high steviol glycoside content (80.6% TSG, Samples A and N) without solid dosage reduction, e.g., 93% TSG for L, which is similar to 90-94% TSG for A; 92% TSG for Q, and 92% TSG for N.

Comparing A, J, and M, one sees that for the same starting material (Samples J, M) with low steviol glycoside content (49.7% TSG), solid dosage minor reduction from 35% (J) to ˜32% (M) leads to greatly increased filtration rates (from 0.4-0.5 g/s for J increased to 1.0-1.1 g/s for M) and elevated product purities (from <86% TSG and 52% Reb A for Sample J increased to 90% TSG and 56% Reb A for Sample M). Furthermore, the filtration rate and product purity of Reb A with solid dosage minor reduction of the present invention are even higher than those from the starting material with a high steviol glycoside content (80.6% TSG for Sample A) without solid dosage reduction, e.g., 1.0-1.1 g/s filtration rate and 56% Reb A for Sample M, which are higher than 0.6-0.7 g/s filtration rate and 42-44% Reb A for Sample A. The product purity of TSG from solid dosage minor reduction is at a similar level to that from the starting material with a high steviol glycoside content (80.6% TSG for Sample A) without solid dosage reduction, e.g., 90% TSG for Sample M, which is similar to 90-94% TSG for Sample A.

Thus, Example 3 shows that a solid dosage minor reduction of a remaining stevia extract starting material that has a low TSG content allows for: (1) faster filtration rate after crystallization; and (2) increased product purities (both TSG % and Reb A %). Compared to using a starting material with a high TSG that corresponds to a high steviol glycoside content, a solid dosage minor reduction during crystallization of a starting material with a low TSG that corresponds to a low steviol glycoside content led to: (1) downstream filtration at a higher filtration rate; (2) similar TSG % in a solid product; and (3) higher Reb A % in solid product.

### Example 4

Starting materials (Samples S, T, U, V, W) for this example were crude stevia extracts as described in [0059]. Their steviol glycoside contents are listed in Table VI.

“The first crystallization” process described in paragraph [0059] was performed on each starting material to obtain pure Reb A as a crystallization product. To that, 330 kg of a starting material as dry solid and 1806 kg of 90% aqueous methanol at 45° C. were added into a crystallizer. The solid dosage was 17.2%. Agitation and cooling were applied. After crystallization, centrifugation was performed to isolate Reb A crystals from the mother liquor (i.e., the first by-product liquid stream, see paragraph [0059]). The wet cake of Reb A crystals was dried to final product.

A reference starting material with >56% Reb A was considered as high quality because of its high Reb A content. Its crystallization was conducted with solid dosage at the baseline level of 17.2%. Its product purities were in quality specification (>95% Reb A). In this example, a starting material with low Reb A content (≤56% Reb A) was considered having low steviol glycoside content.

Table VI provides a summary of the effects on product purity (Reb A %) from low Reb A contents (<56% Reb A) in various starting materials that were subjected to solid dosage minor reduction to obtain pure Reb A.

TABLE VI
Low Reb A Starting Materials Subjected to Solid Dosage Minor
Reduction in the 1st Crystallization to Obtain Pure Reb A

| | Starting | Starting | Solid | Product | Product | Product | Product | |
|---|---|---|---|---|---|---|---|---|
| | Material | Material | Dosage, | Yield, % | Reb A, % | TSG, % | Reb A | Product TSG |
| Sample | Reb A, % | TSG, % | % | (db) | (db) | (db) | Recovery % | Recovery % |
| S | 51.5 | 79.3 | 17.2 | 32 | 93.5 | 97.9 | 58 | 39 |
| T | 54.1 | 85.3 | 15.4 | 33 | 96.0 | 99.1 | 59 | 38 |
| U | 46.8 | 79.6 | 14.9 | 26 | 95.7 | 98.6 | 54 | 32 |
| V | 51.6 | 82.9 | 15.3 | 30 | 96.5 | 99.6 | 57 | 36 |
| W | 47.9 | 81.7 | 14.9 | 27 | 95.2 | 98.5 | 53 | 32 |

In Table VI, the product purity (93.5% Reb A) from Sample S, a low Reb A starting material (51.5% Reb A), was below the product specification (>95% Reb A) with the solid dosage kept the same value at 17.2% as for the high Reb A starting material (>56% Reb A). A comparison of Samples T, U, V, and W to Sample S shows that a solid dosage minor reduction from 17.2% (S) to 14.9-15.4% (T, U, V, W) for low Reb A starting materials resulted in remarkable purity increases in final crystal products, i.e., from 93.5% Reb A (S) increased to 95.2-96.5% Reb A (T, U, V, W).

### Example 5

A starting material comprising Reb M (sample composition is as follows: Reb O: 10.34%, Reb D: 30.62%, Reb N: 8.14%, Reb M: 23.20%, Reb A: 5.48%, Reb B: 2.63%, Stevioside: 0.42%, and Total Steviol Glycoside: 80.83%; all values are reported dry basis) with a solid dosage of 18.42% was obtained by dissolving the starting material in 49% methanol solvent (balance is DI water). The starting material was subjected to the following processes: (1) crystallization and filtration at a temperature of 55° C.; (2) solid dosage minor reduction at 55° C. followed by crystallization and filtration at a temperature of 55° C.; and (3) solid dosage minor reduction at 55° C. followed by crystallization and filtration at a temperature of 75° C. The results appear in Table VII, which show that increasing the temperature under which crystallization and filtration occur significantly improves filtration time after solid dosage minor reduction.

TABLE VI
Increased Temperature During Crystallization and Filtration.

| | | | | Filtration |
|---|---|---|---|---|
| | | Crystallization | Filtration | Time |
| Experiment | % Solids | Temp (° C.) | Temp (° C.) | (Second) |
| 1 | 18.42 (starting | 55 | 55 | 300 |
| | material) | | | |
| 2 | 14.42 (reduced | 55 | 55 | 270 |
| | solid dosage) | | | |
| 3 | 14.42 (reduced | 75 | 75 | 65 |
| | solid dosage) | | | |

We claim:

1. A method for making a crystallization product comprising:
a) obtaining a starting material, wherein the starting material comprises solids and a solvent, wherein the solids comprise one or a plurality of steviol glycosides, and the starting material is at a solid dosage of X wt. %, wherein
$$X = \frac{weight\_solid}{weight\_solid + weight\_solvent} \times 100 ,$$
weight_solid=combined weight of all solids in the starting material, and weight_solvent=total weight of the solvent in the starting material;
and the starting material has any one, two or all three of (i) a total composition of steviol glycosides of less than or equal to 85% on a dry weight basis, (ii) Reb A in an amount of less than or equal to 56% on a dry weight basis, and (iii) Reb M in an amount of minimum 20% on a dry weight basis;
b) reducing the solid dosage of the starting material to form a material with a reduced solid dosage, wherein the reduced solid dosage is Y wt. % wherein Y is between 70% and 98% of X;
c) after step (b) subjecting the material with a reduced solid dosage to a crystallization process; and
d) obtaining a crystallization product comprising one or more crystals.

2. The method according to claim 1, wherein Y is between 75% and 95% of X.

3. The method according to claim 1, wherein X is between 14 and 36.

4. The method according to claim 1, wherein X is between 32 and 36.

5. The method according to claim 1, wherein X is between 14 and 20.

6. The method according to claim 1, wherein X is between 17 and 19.

7. The method according to claim 1, wherein the starting material comprises a plurality of steviol glycosides.

8. The method according to claim 1, wherein in the starting material, the plurality of steviol glycosides comprises at least one of Reb A, Reb B, Reb C, Reb D, Reb F, dulcoside A, rubusoside, stevioside, steviolbioside, and Reb M.

9. The method according to claim 1, wherein in the starting material, the plurality of steviol glycosides comprises each of Reb A, Reb B, Reb C, Reb D, Reb F, dulcoside A, rubusoside, stevioside, and steviolbioside.

10. The method according to claim 1, wherein in the starting material, the steviol glycoside content due to steviol glycosides other than Reb A, Reb B, Reb C, Reb D, Reb F, dulcoside A, rubusoside, stevioside, and steviolbioside is less than 5.0% or is 0% on a dry weight basis.

11. The method according to claim 1, wherein in the starting material, the steviol glycoside content due to steviol glycosides other than Reb A, Reb B, Reb C, Reb D, Reb F, dulcoside A, rubusoside, stevioside, and steviolbioside is less than 2.0% or is 0% on a dry weight basis.

12. The method according to claim 1, wherein the starting material comprises Reb M.

13. The method according to claim 1, wherein the starting material comprises a crude stevia extract.

14. The method according to claim 1, wherein the crystallization process is a second crystallization process and the crystallization product is a second crystallization product, and the method further comprises
subjecting a stevia extract to a first crystallization process thereby obtaining a first crystallization product and a by-product liquid stream as a mother liquor;
spray drying the by-product liquid stream, thereby obtaining a set of remaining steviol glycosides;
and using the set of remaining steviol glycosides as said starting material.

15. (canceled)

16. The method of claim 1, wherein the starting material has both (i) the total steviol glycoside amounts of less than or equal to 73% on a dry weight basis, and (ii) the Reb A is an amount of less than or equal to 56% on a dry weight basis.

17. The method of claim 1, wherein the total composition of steviol glycosides is less than 80%.

18. The method of claim 1, wherein the total composition of steviol glycosides is less than 73%.

19. The method of claim 1, wherein in the starting material Reb M is present in an amount of minimum 20% on a dry weight basis.

20. The method of claim 1, wherein step (c) is conducted at a temperature of greater than 55° C.

21. The method of claim 1, wherein step (d) comprises filtering and said filtering occurs at a temperature of greater than 55° C.

22. (canceled)

23. (canceled)

24. (canceled)
